(19) United States
(12) Patent Application Publication
(10) Pub. No.: US 2014/0378940 A1
(43) Pub. Date: Dec. 25, 2014

(54) Anti-nociceptive apparatus

(71) Applicant: Choon Kee Lee, Denver, CO (US)

(72) Inventor: Choon Kee Lee, Denver, CO (US)

(21) Appl. No.: 13/924,593

(22) Filed: Jun. 23, 2013

(51) Int. Cl. A61M 5/42

(52) U.S. Cl. CPC A61M 5/422
USPC 604/506; 604/112

(57) ABSTRACT

The present invention presents an apparatus and methods to generate and deliver circumferential mechanical vibrations to a hypodermic needle penetration site of a human body to reduce pain of needle prick by activating inhibitory neuronal mechanisms for pain perception. The apparatus comprises a detachably disposable proximal end that contacts with a tissue of a recipient and encircles a needle penetration site, a distal end that is configured as a conduit for electric power to the apparatus and a longitudinally tubular handle assembly that is connected to both proximal and distal ends and that houses a vibration assembly and a control and power assembly. The vibration assembly generates and delivers resonant vibrations to the recipient through the proximal end. A needle penetrates the recipient's tissue circumferentially surrounded by vibrations transmitted by the proximal end.

### CROSS REFERENCE TO RELATED APPLICATIONS

Attached please refer to the Information Disclosure. Statement for the cross reference to related applications.

### STATEMENT REGARDING FEDERALLY SPONSORED RESEARCH OR DEVELOPMENT

The present invention is not a federally sponsored research or development.

### TECHNICAL FIELD

The present invention relates generally to the field of hypodermic injection of an agent for medical purpose. More specifically, the present invention provides an apparatus and methods to reduce pain and discomfort associated with an entry of a needle and an injectable agent into tissue.

### BACKGROUND OF THE INVENTION

Injection of an agent into cutaneous and muscle tissues through a needle prick disrupts mechanical and chemical stability of the tissue and initiates a series of electrophysiological and biochemical cascade in the local tissue environment and in free nerve endings of nociceptive primary afferent nerve fibers embedded in the tissue. Cationic channels of the free nerve endings are activated, dependent on biophysical properties of both the needle prick and injected agent. Once voltage gated Na+ channels are activated, membrane depolarization of the nociceptor is propagated, resulting in release of intracellular Ca++. The increase in Ca++ concentration mediates cellular and microenvironmental changes to sensitize nociceptors of the free nerve endings. Furthermore, cells that are disrupted by needle prick could release membrane fatty acids which convert to prostaglandins. Increase in prostaglandins could intensify nociceptive response of the free nerve endings, which translates into intensified painful sensation by a subject.

The majority of the nociceptive signals generated by the free nerve endings are transmitted via both A-delta and C nerve fibers to superficial dorsal horn of the spinal cord. A-delta nerve fibers are responsible for initial sensation of sharp localized pain and C fibers are responsible for so-called second pain of burning and bruised feeling over a wider area than perceived by the A-delta fibers. A-delta fibers are known to be sensitized by intense heat, and high intensity and prolonged activation of C fibers are known to perpetuate the sensitization cycle of C fibers by producing ligands acting on release of pro-inflammatory molecules. At the spinal cord, both A-delta and C-fibers produce glutamate that is a key molecule for transmission of sensation of pain. Postsynaptic nociceptive input then travels upward from the spinal cord to various parts of brain.

There are inhibitory neuronal signals arising from various parts of the brain that descend in the spinal cord to modulate nociception. Descending inhibitory signals may be activated by external factors including stimulation on peripheral or central nervous system. In addition, there are ascending inhibitory signals, albeit minor, arising from parts of the brain. Descending inhibitory signals come to various neuronal structures of the dorsal horn of the spinal cord where downward postsynaptic changes inhibit nociceptive responses. It is believed that in human subjects the descending inhibitory signals can be physically activated by acupuncture, transcutaneous electric nerve simulation (TENS), vibration, dorsal column stimulation and deep brain stimulation.

Vibration is one of peripheral stimulation methods to reduce nociception, which include TENS, acupuncture, acupuncture-like TENS, electroacupuncture and acupressure. Exact mechanisms of analgesia induced by vibration have not been clarified yet but it is believed to be related to activation of A-beta primary afferent nerve fibers that inhibit segmental neurons of the dorsal horn of the spinal cord. It is also proposed that vibration stimulates both high-threshold A-beta fibers and A-delta fibers, which activates the descending inhibitory signals to suppress the dorsal horn neurons. Clinically, both TENS and vibration have been shown to reduce acute and chronic pain conditions, including low back pain, acute orofacial pain, causalgia, pain associated with vaginal delivery of baby and arthritic pain. In particular, vibration of cutaneous tissue of patients has been shown to reduce pain associated with needle prick and injection of agents into the tissue, thereby reducing requirement of anesthetic agents for minor procedures on skin and its appendages.

Various frequencies have been studied for vibration induced analgesia, ranging from 20 Hz to 300 Hz with a varying degree of effectiveness on analgesia. Additional issues of vibration such as duration, amplitude and effective area and depth under vibration have not been studied for its comparative effectiveness except that it appears that analgesia is achieved best in an area directly under vibration. Shortcomings of vibration are short duration of effects and potential development of tolerance over repetitive uses.

Needle-free injection systems using high-pressure jet-stream have been developed over a few years to reduce discomfort of needle prick necessary for injecting agents into tissue. However, needle-free injection disrupts mechanical and chemical stability of the tissue, which initiates similar electrophysiological and biochemical responses in nociceptive primary afferent nerve fibers to needle-based injection systems. Diffuse but limited dispersion from a site of entry of pressured jet-stream of the needle-free system inside the tissue along a longitudinal injection path may be the only advantage of the needle-free system to the needle-based system that produces a radially globular expansion of an injected agent from a tip of a needle inserted in the tissue. It is conceivable that globular expansion of the injected agent, compared to the longitudinally diffuse dispersion of the agent, may exert a more outward pressure per an area of the tissue, thereby disrupting a larger amount of mechanical connection of the tissue. However, one major drawback of the needle-free injection system is a risk of contamination of injection nozzle by recipient's tissue fluid that may emanate from an entry site of injection of the recipient. Unless each device is used only once for each recipient, it poses a significant hazard of transmission of potentially infectious agents such as hepatitis virus or human immunodeficiency virus (HIV) to other recipients receiving injection using the same device. Disposable needle-free injection systems would be available yet their cost-effectiveness cannot be compared favorably to simple disposable syringes and steel needles.

Intensity of nociception, i.e., pain sensation, associated with conventional hypodermic injection of an agent may be ameliorated by limiting extent of mechanical and chemical disruption of a target tissue and by activating descending inhibitory signals. Thinner and shorter hypodermic needles with a more acute angle of bevel may reduce the extent of mechanical disruption of the tissue. Stimulation of an injection site by vibration is one of available methods to activate the descending inhibitory signals. Successful implementation of vibration for achieving analgesia during the needle-based injection would require generation of a vibration field surrounding both a needle penetration site and a tissue infiltration site of an injected agent for an adequate length of time, adequate and redundant activation of primary afferent nerve fibers and fast diffusion of the injected agent from the tip of a needle to adjacent tissues. Yet the foremost importance should be given to a reproducible method of fail-safe delivery of an agent to a recipient without a risk of contamination by biologic fluids.

### SUMMARY OF THE INVENTION

To achieve on-site placement of vibration surrounding a needle penetration site of a recipient and a tissue infiltration site of an injected agent and to eliminate a risk of cross-contamination of other recipients by a contaminated apparatus by biologic fluids emanating from a needle penetration site of a recipient, the current apparatus comprises a detachably disposable vibration tip located at the proximal end, a distal end configured as a conduit for electric power and a longitudinally tubular handle assembly housing a vibration assembly and a control and power assembly. A distal part of the vibration tip is configured to be releasably and slidably coupled with a longitudinally cylindrical vibration resonance enclosure of the handle assembly. A proximal part of said vibration tip contacts a recipient's skin and is configured to provide a circumferential field of vibration surrounding a needle penetration site. The vibration assembly of the handle assembly generates vibrations that are resonated by the vibration resonance enclosure located at a proximal end of said vibration assembly. The control and power assembly comprises a battery, an electronic circuit board, a switch and an instrument panel, which are electrically connected with each other and are disposed in and about the handle assembly. The distal end is configured for replacing or recharging a battery of the control and power assembly.

In one embodiment, the detachably disposable vibration tip, provided as one or a plurality of operating devices having one or a plurality of configurations, comprises a distal elongated round body and a proximal ring portion connected to said elongated round body at an angle. There is provided a central tubular space for a length in the elongated round body along the longitudinal axis, which opens distally to a junctional surface of said elongated round body. The ring portion of the vibration tip, provided in one or a plurality of configurations, comprises a circumferential rim and a neck that connects the ring portion to the elongated round body. The circumferential rim is configured in a range of cross-sectional thickness, radius and elasticity. The ring portion is configured to contact with and to encircle an area of a tissue that is penetrated by a needle and to deliver vibrations to said area of needle penetration.

In one embodiment, the handle assembly, provided as one or a plurality of operating devices having one or a plurality of configurations, comprises a vibration assembly and a control and power assembly, arranged in tandem along the longitudinal axis inside said handle assembly. One of the configurations of the handle assembly includes a compartmentalized tubular structure that is trapezoidally round along the circumferential axis. In one embodiment, the vibration assembly is housed in a proximal compartment and the control and power assembly in a distal compartment of said handle assembly. In between of the compartments, there is provided a space that is filled with one or a plurality of vibration absorbing materials. In another embodiment, the handle assembly is equipped with one or a plurality of means to shield an operator's hand from an electromagnetic field generated by the vibration assembly. One of the means includes coating of an inner surface of said handle assembly by heavy metals such as copper or aluminum.

In one embodiment, the vibration assembly, provided as one or a plurality of operating devices having one or a plurality of mechanical configurations, comprises a vibration generator and a vibration resonance enclosure which is connected to the vibration generator. The vibration resonance enclosure, provided in one or a plurality of mechanical configurations including a cylindrically tubular configuration, is located proximal to the vibration generator along the longitudinal axis and protrudes longitudinally through a junctional surface of the proximal end of the handle assembly. A proximal portion of the vibration resonance enclosure is configured to be releasably and closely insertable in the inner central tubular space of the elongated round body of said vibration tip. A distal end of the vibration resonance enclosure is configured to be attached to the vibration generator and transmits vibrations to said vibration resonance enclosure. The vibrations then are transmitted from the vibration resonance enclosure to the elongated round body of the vibration tip and to the ring portion of the vibration tip.

In one embodiment, vibration is generated by an electromagnetic voice coil actuator with a moving coil, provided in one or a plurality of configurations, releasably and axially inserted in one or a plurality of cylindrical permanent magnets of said voice coil actuator. In a second embodiment, vibration is generated by an electromagnetic solenoid coil, provided in one or a plurality of configurations, releasably and axially inserted in one or a plurality of cylindrical permanent magnets. In another embodiment, vibration is produced by a vibratory electromagnetic motor provided as one or a plurality of mechanical configurations including an eccentric mass rotary motor or by an electromagnetic disc vibrator. A proximal end of the moving coil of the voice coil actuator, the vibratory rotary motor, or the disc vibrator is attached to a vibration cone which in turn is attached to the distal end of the vibration resonance enclosure. The vibration cone is configured as diaphragm in a range of thickness and pliability.

In one embodiment, the vibration resonance enclosure provides resonance which amplifies vibration in a certain range of frequencies generated by the vibration generator. One of the configurations of the vibration resonance enclosure provides a natural frequency of said resonance matched to a frequency range from 20 Hz to 300 Hz. The vibration resonance enclosure may or may not have a cylindrically tubular space inside said enclosure. A vibration resonance enclosure without the cylindrically tubular space is configured as a solid cylinder to which the vibration is directly transmitted and resonated.

In one embodiment, a moving coil of a voice coil actuator produces electromagnetic vibration in one or a plurality of frequencies ranging from 20 Hz to 20 kHz and of one or a plurality of amplitudes. In another embodiment, the voice coil actuator simultaneously generates vibrations of multiple frequencies. Concurrent generation of vibrations in multiple frequencies is meant to cover a wide range of nociceptive primary afferent nerve fibers which may have individually distinctive activation thresholds to different frequencies for activating inhibitory signals. Vibration amplitude is provided as adjustable to penetration depth of needle into tissue and to volume of injectable agent. A deeper penetration of a needle into a tissue and a larger volume of an injectable agent require a wider and deeper vibration field to sufficiently encompass the area of the injection, compared to a shallow penetration and to a smaller injection volume. Force of vibration is proportional to amplitude of vibration, which suggests that a higher amplitude is required to generate a larger force of vibration to cover a larger three-dimensional volume of a tissue that needs to be vibrated. Electromagnetic disc vibrator is also provided in one or a plurality of frequencies and with one or a plurality of amplitudes. The disc vibrator also is configured to simultaneously generate vibrations of multiple frequencies. Vibratory eccentric mass rotary motor is provided in frequency that is variable.

In one embodiment, the control and power assembly, comprising a electronic control unit and a power source, is housed in the handle assembly and connected electrically to the vibration generator. The power source includes one or a plurality of replaceable or rechargeable batteries and is electrically connected to the electronic control unit. The electronic control unit is provided as one or a plurality of electronic configurations, which comprises an electronic circuit board, a switch and an instrument panel and which controls an electric current to the vibration generator and modulates both frequency and amplitude of vibration. Both the electronic circuit board and the power source are housed in a compartment of the handle assembly, which is located distal to the vibration assembly compartment of said handle assembly and is enclosed by one or a plurality of vibration absorbing materials.

In one embodiment, an operator, using one hand, places the ring portion of the vibration tip of the apparatus with a firm pressure to a sterilized skin of a recipient and switches on said apparatus to provide the recipient with vibrations. The operator, using the other hand, pushes in a needle of a syringe in the middle of an encircled area of a tissue by the rim of the ring portion of the vibration tip of said apparatus and delivers an injectable agent into the tissue. Upon completion of the injection, the needle of the syringe is removed first, followed by switching off said apparatus and discarding the vibration tip by pulling out said vibration tip from the proximal end of the handle assembly. A new vibration tip then is installed for a next recipient.

### BRIEF DESCRIPTION OF THE DRAWINGS

Overview shows a schematic example of the apparatus of the present invention.

FIG. 1 shows an schematic example of itemized components of the apparatus.

FIG. 2 shows a schematic two-dimensional example of individual devices of the apparatus: FIG. 2A represents a profile view; FIG. 2B shows a frontal view; FIG. 2C shows a frontal view of a handle assembly; FIG. 2D shows a frontal view of a detachable vibration tip.

FIG. 3 shows a schematic example of the vibration tip: FIG. 3A and 3B represent an example of individual parts of the vibration tip; FIGS. 3C-3F show examples of various configurations of the vibration tip.

FIG. 4 shows a schematic example of the handle assembly; FIG. 4A represents individual components of a vibration assembly and a control and power assembly housed in the handle assembly; FIG. 4B shows a vibration generator of a voice coil actuator or a solenoid coil; FIG. 4C shows a placement of a vibratory eccentric mass rotary motor in a proximal compartment of the handle assembly; FIG. 4D shows an example of a vibratory eccentric mass rotary motor; FIG. 4E shows an example of an electromagnetic disc vibrator.

FIG. 5 shows a schematic example of a longitudinally cross-sectional view of the handle assembly.

### DETAILED DESCRIPTION OF THE DRAWINGS

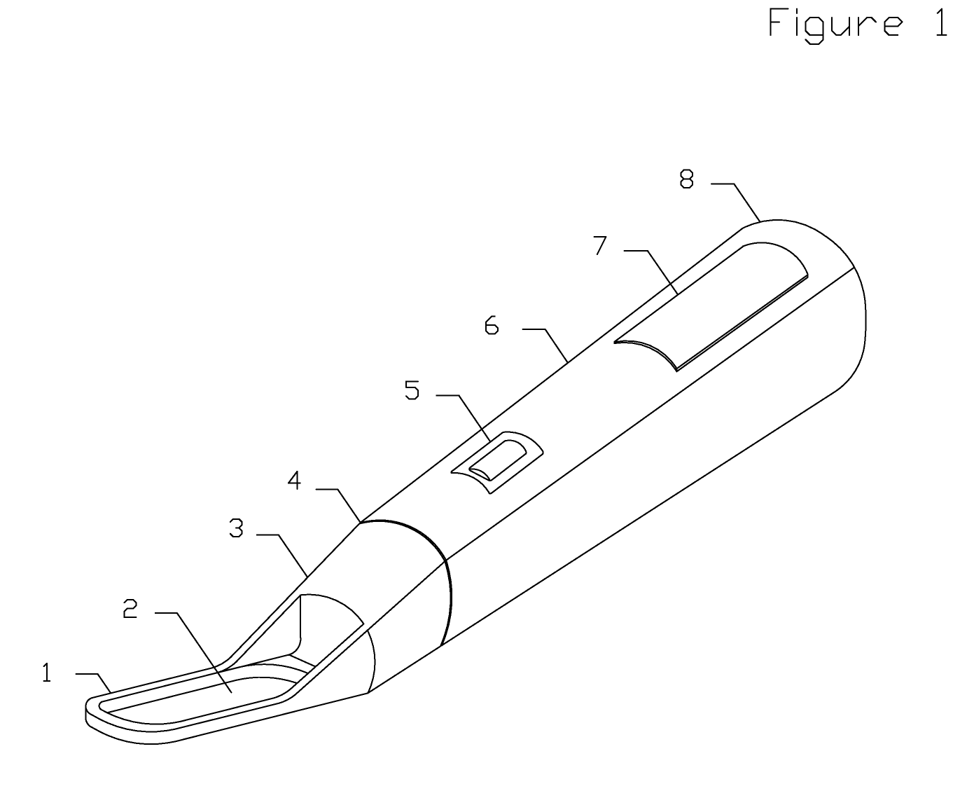

As described below, the present invention provides a vibration analgesia apparatus and methods of use. It is to be understood that the descriptions are solely for the purposes of illustrating the present invention, and should not be understood in any way as restrictive or limited. Embodiments of the present invention are preferably depicted with reference to FIGS. 1 to 5, however, such reference is not intended to limit the present invention in any manner. The drawings do not represent actual dimension of devices, but illustrate the principles of the present invention.

The overview shows a schematic three-dimensional illustration of an example of the apparatus. FIG. 1 shows an itemized view of the schematic example of individual parts of the apparatus. A vibration tip comprises a ring portion 1 encircling a planar space 2, an elongated round body 3 that joins a handle assembly 6 at a joint 4. The handle assembly 6 joins the vibration tip at the junction 4 and distally ends at a distal end 8. A control switch 5 and an instrument panel 7 are placed longitudinally on an outer surface of the handle assembly 6.

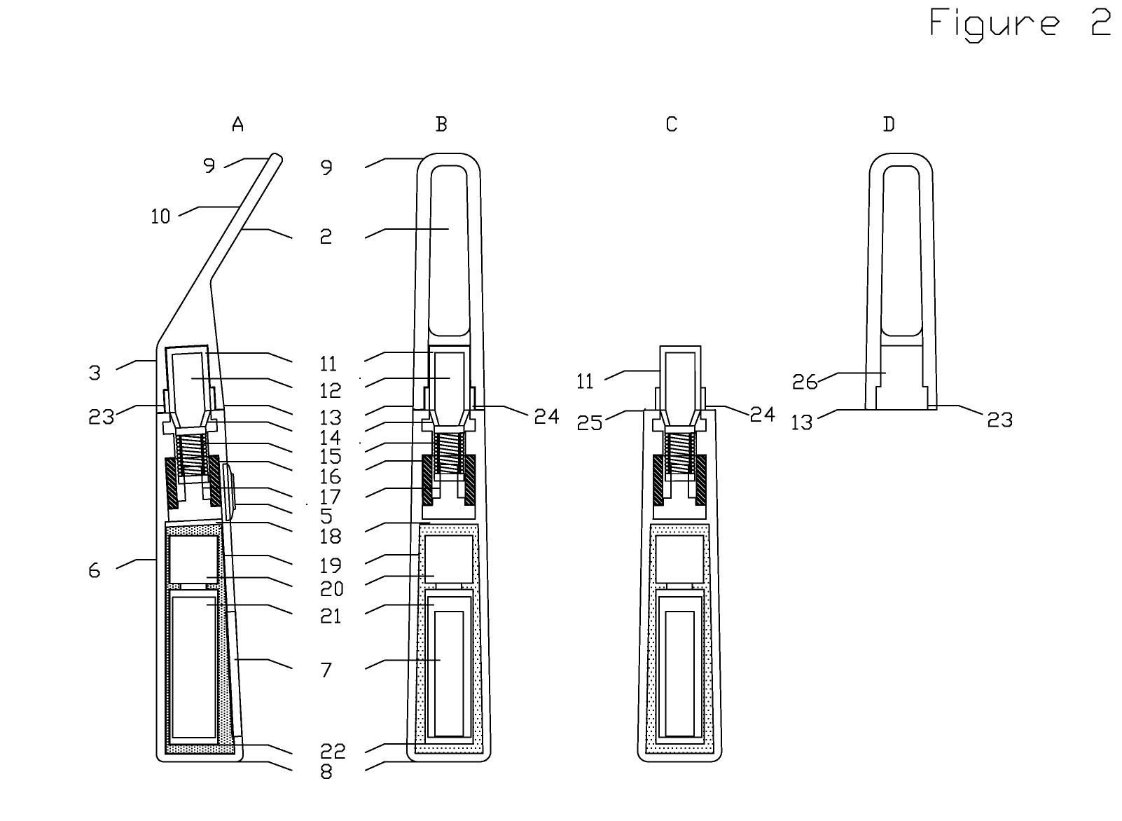

FIG. 2 shows a schematic two-dimensional example of individual devices of the apparatus. FIG. 2A represents a profile view and FIG. 2B shows a frontal view. FIG. 2C shows a frontal view of a handle assembly and FIG. 2D shows a frontal view of a detachable vibration tip. The vibration tip, provided in one or a plurality of configurations, has a proximal end 9 proximally bordering a planar rim 10 and a distal end 13 of the elongated round body 3. The planar rim 10 encircles the planar space 2 which provides an area for a needle penetration. As depicted in FIGS. 2C and 2D, the elongated round body 3 of the tip has an inner central tubular portion 26 in and out of which a cylindrical vibration resonance enclosure 11 of the handle assembly 6 reversibly slides. There is provided a flange portion 24 surrounding the vibration resonance enclosure 11, which is irreversibly attached to a proximal end 25 of the handle assembly 6 and to the vibration resonance enclosure 11. The flange portion 24 provides reversible tight coupling between a flange receptacle portion 23 of the vibration tip and the vibration resonance enclosure 11.

In FIGS. 2A and 2B, the vibration resonance enclosure 11 of the handle assembly 6 is illustrated as enclosing a resonant vibration chamber 12 which is connected to a voice coil actuator 15 via a vibration cone 14. The voice coil actuator 15 is surrounded by a cylindrical permanent magnet 16 which is irreversibly held in place by a cylindrical central magnet holder 17 of the handle assembly 6. The vibration assembly of the apparatus comprises the vibration resonance enclosure 11 with the resonant vibration chamber 12, the vibration cone 14, the voice coil actuator 15, the cylindrical permanent magnet 16 and the cylindrical central magnet holder 17. The voice coil actuator 15 is releasably inserted in the cylindrical permanent magnet 16 and receives electricity from a control and power assembly of the handle assembly 6. The cylindrical permanent magnet 16 is immovably fixed to a surrounding vibration generator housing cylinder and the voice coil actuator 15 axially moves back and forth inside said magnet 16 along the longitudinal axis dependent on electromagnetic polarity provided by the control and power assembly. Vibrations generated by axial movements of the voice coil actuator 15 are transmitted to the resonant vibration chamber 12 through the vibration cone 14. The resonant vibration chamber 12 amplifies vibrations of a range of frequencies and transmits the resonated vibrations to the elongated round body 3 of the vibration tip. The elongated round body 3 then transmits the vibrations to the planar rim 10 which delivers the vibrations to the encircled space 2.

In an example of one configuration, illustrated in FIGS. 2A and 2B, the control and power assembly of the handle assembly 6 comprises the electronic switch 5 close to the proximal end 25, the instrument panel 7 close to a distal end 8 of the handle assembly 6, an electronic circuit board 20 and a battery 21. Both the electric switch 5 and instrument panel 7 are located on an outer surface of the handle assembly. Both the electronic circuit board and battery 21 are enclosed by a cylindrical compartment 19. The cylindrical compartment 19 is located distally to and separated from the vibration assembly by a space 18. An inner surface of the cylindrical compartment 19 is lined by one or a plurality of vibration absorbing materials. A longitudinal axis of the cylindrical compartment 19 is configured not to be coaxial with a longitudinal axis of the vibration assembly, to reduce sympathetic resonance. The battery 21 is accessible through the distal end 8 of the handle assembly and a distal end 22 of a battery enclosure. All devices of the control and power assembly are electrically connected with each other and with the voice coil actuator 15. The battery 21 is provided as one or a plurality of devices including replaceable or rechargeable battery. For a battery recharge system, the distal end 22 of the battery enclosure provides a connection port in one or a plurality of configurations for recharging battery, including a plug receptacle or a secondary coil for induction. The electronic switch 5, provided as one or a plurality of operating devices having one or a plurality of mechanical and electronic configurations, turns on and off the vibration generator and varies frequencies and amplitudes of vibration by a plurality of pre-set numbers of push-to-make and push-to-break actions on said switch.

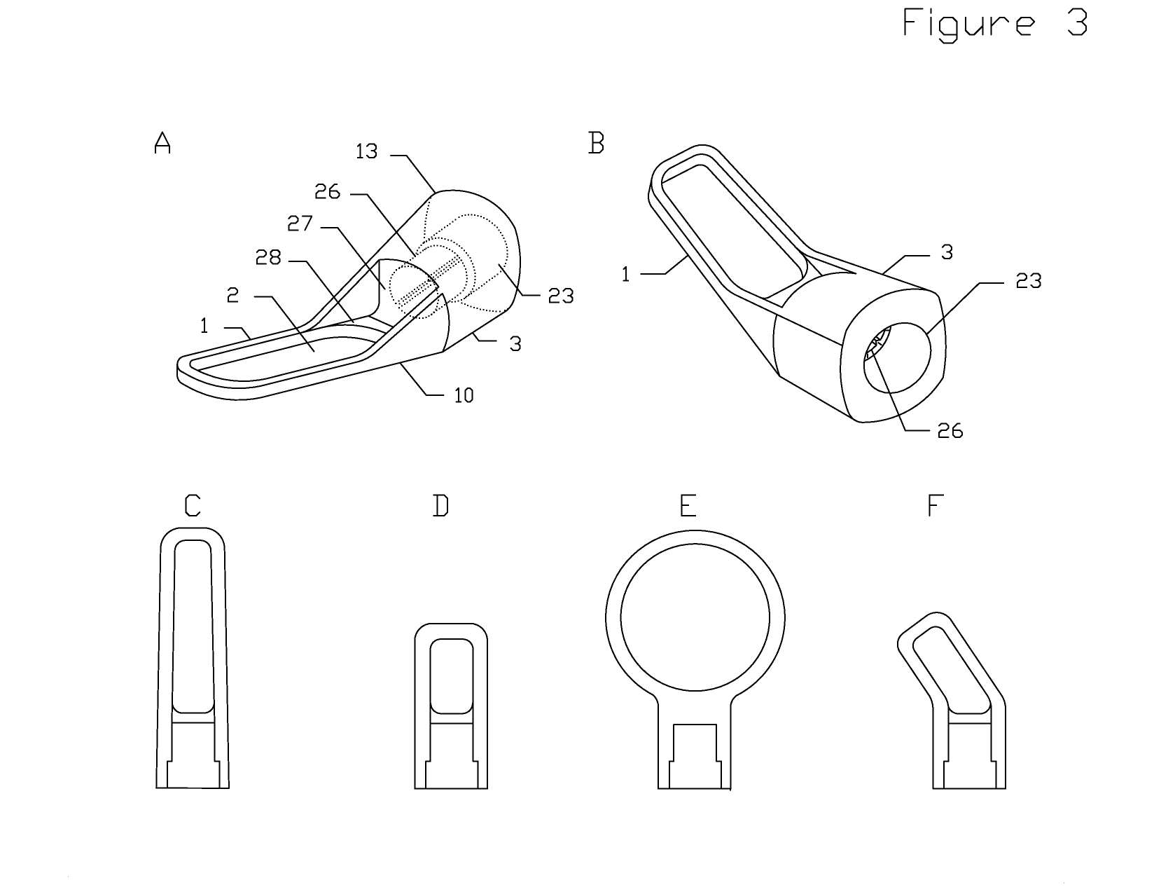

FIG. 3 shows a schematic example of the vibration tip. FIGS. 3A and 3B represent an example of individual parts of the vibration tip. FIGS. 3C-3F show examples of various configurations of the vibration tip. Depicted in FIGS. 3A and 3B, the ring portion 1, provided in one or a plurality of configurations, comprises the planar rim 10 and a connecting neck portion 28 to a proximal end 27 of the elongated round body 3 at an angle. One of the configurations of the rim 10 includes a cross-sectionally rectangular and longitudinally flat rim from the neck portion to the proximal end of the rim, which is configured to contact with a tissue of a recipient and to deliver circumferential vibrations surrounding the space 2. The rim 10 is made of one or a plurality of polymeric materials and is provided in a range of cross-sectional thickness, circumference and elasticity. The elongated round body 3, provided in one or a plurality of configurations including a tapered cone configuration, has the inner central tubular portion 26 and the tubular flange receptacle 23 which opens to the distal end 13 of said body. The inner central tubular portion 26 is configured to have a means to secure the vibration resonance enclosure 11, which includes a pair of linear threads protruding from an outer surface of said inner central tubular portion. Referring to FIG. 2B, the inner central tubular portion 26 and the tubular flange receptacle 23 are reversibly and insertably coupled with the vibration resonance enclosure 11 and the flange 24 of the handle assembly 6. Various configurations of the ring portion 1 of the vibration tip are illustrated as examples in FIGS. 3C-3F. FIG. 3C shows an elongated rectangular rim, FIG. 3D shows a short rectangular rim, FIG. 3E shows a circular rim and FIG. D shows an angled rim at the neck portion.

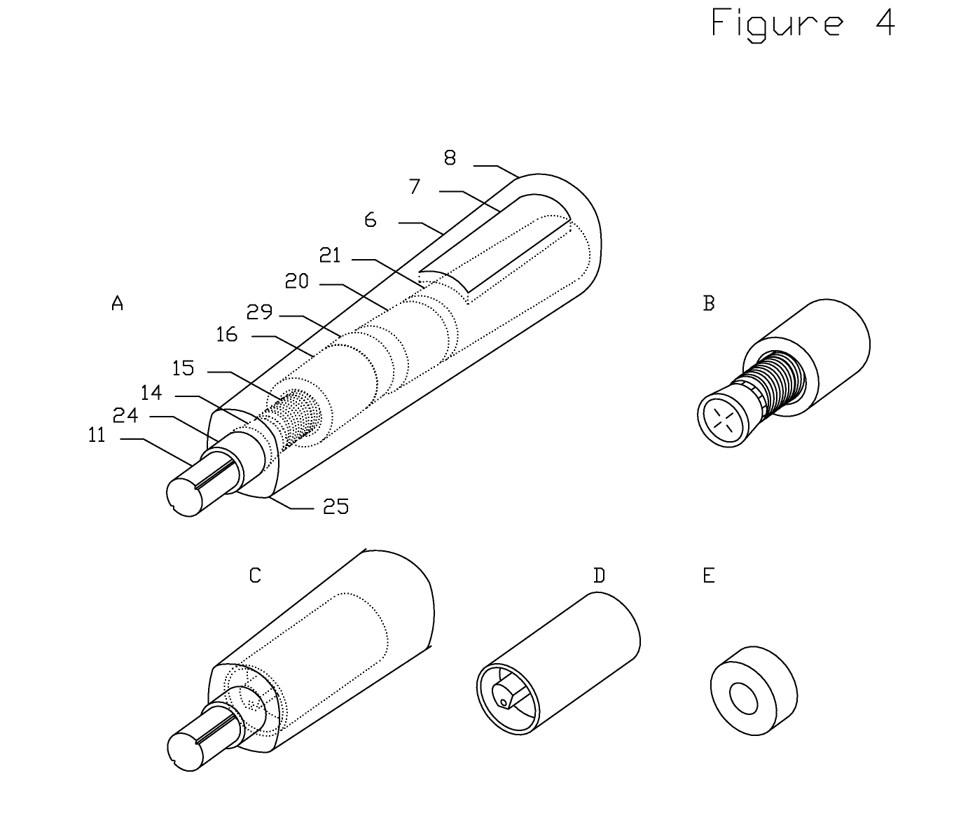

FIG. 4 shows a schematic example of the handle assembly and a layout of the devices inside said handle assembly. FIG. 4A represents individual devices of the vibration assembly and the control and power assembly. Referring to FIG. 3A, the vibration resonance enclosure 11 is configured to have a means to be paired with the linear threads of the inner central tubular portion 26 of the vibration tip, including a pair of linear notches carved in an outer surface of said vibration resonance enclosure. The handle assembly 6 is bordered proximally by the proximal end 25 and distally by the distal end 8. Inside the handle assembly 6, devices are longitudinally arranged in tandem, including the vibration cone 14, the voice coil actuator 15, the cylindrical permanent magnet 16, a cylindrical central magnet holder base 29, the electronic circuit board 20 and the battery 21. The instrument panel 7 is shown, attached on the outer surface of the handle assembly. FIG. 4B shows a schematic example of a voice coil actuator or a solenoid coil for generating vibrations. FIG. 4C shows a in-situ placement of a vibratory eccentric mass rotary motor in a proximal compartment of the handle assembly. FIG. 4D shows an example of a vibratory eccentric mass rotary motor and FIG. 4E shows an example of an electromagnetic disc vibrator which can similarly be installed in the proximal compartment for the vibration assembly.

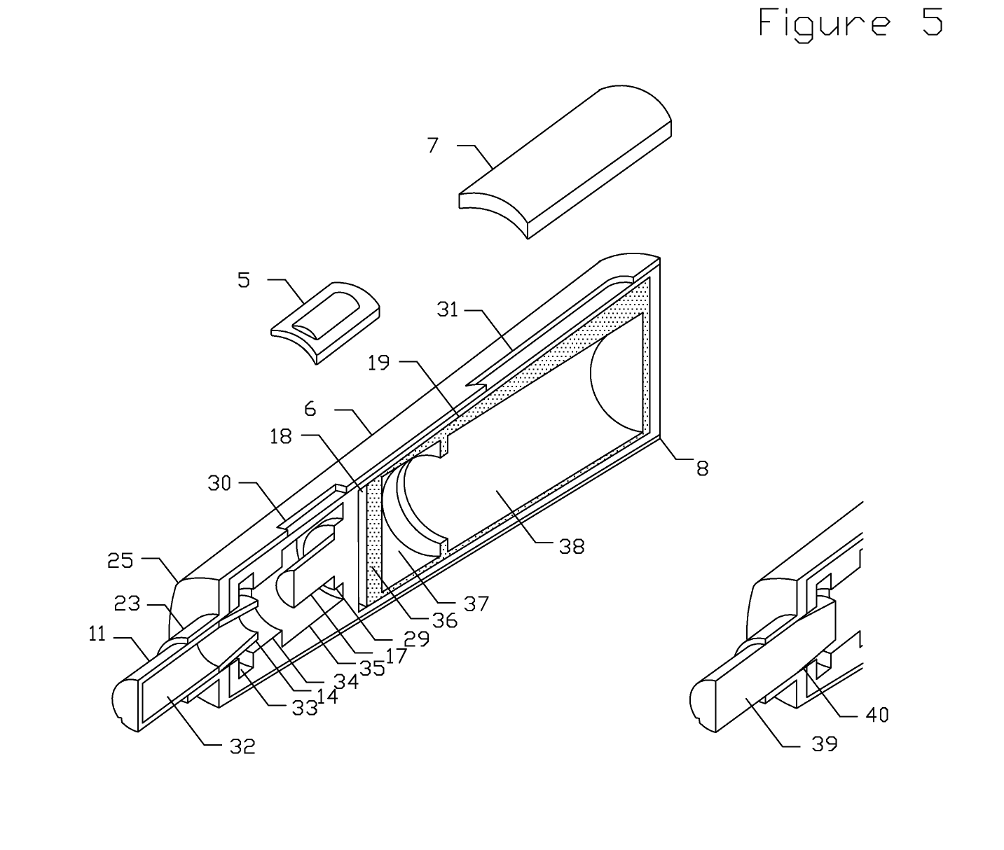

FIG. 5 shows a schematic example of a longitudinally cross-sectional view of an inner layout of compartments of the handle assembly. The electronic switch 5 and the instrument panel 7 are fixedly inserted to a switch recess 30 and an instrument panel recess 31, respectively, both of which are located on the outer surface of the handle assembly 6. A cutaway view 32 of the vibration resonance enclosure 11 shows a cylindrically tubular resonant space which is connected to the vibration cone 14. There is provided a tubular recess 33 circumferentially surrounding the vibration cone 14 to allow unimpeded vibration of said cone 14. Referring to FIG. 2A, the voice coil actuator 15 is longitudinally inserted in a tubular voice coil actuator enclosure 34. The cylindrical permanent magnet 16 is fixedly inserted in between of a tubular magnet enclosure 35 and the cylindrical central magnet holder 17 axially connected to the cylindrical central magnet holder base 29. The cylindrical compartment 19, provided in one or a plurality of configurations, includes a configuration of a two-compartment structure with a proximal tubular electronic circuit board enclosure 37 and a distal tubular battery enclosure 38. There is provided a connecting channel between the tubular electronic circuit board enclosure 37 and the tubular battery enclosure 38 for electric connection. Both the enclosures 37 and 38 are enveloped by a vibration absorbing filler 36 inside the cylindrical compartment 19. In one embodiment, the vibration resonance enclosure 11 is configured as a solid cylinder 39 which is separate by a gap 40 from the proximal end 25 of the handle assembly to reduce transmission of vibrations to a tubular wall of the handle assembly. In one embodiment, the inner surface of the compartments of the handle assembly is covered with one or a plurality of electromagnetic field-shielding materials such as copper or aluminum to reduce exposure of an operator's hand to the electromagnetic field.

It is to be understood that the aforementioned description of the apparatus and methods is simple illustrative embodiments of the principles of the present invention. Various modifications and variations of the description of the present invention are expected to occur to those skilled in the art without departing from the spirit and scope of the present invention. Therefore the present invention is to be defined not by the aforementioned description but instead by the spirit and scope of the following claims.

What is claimed is:

1. An anti-nociceptive apparatus, comprising:
a vibration means located at a proximal end, detachably connected to a handle assembly along a longitudinal axis;
the vibration means, provided in one or a plurality of configurations, which encircles a needle penetration site of a recipient's tissue by contact with said tissue, which receives vibrations from the handle assembly and delivers circumferential vibrations to said tissue; and
the handle assembly, provided as one or a plurality of operating devices having one or a plurality of mechanical and electronic configurations, which is releasably connected with the vibration means, which generates and controls vibrations and which transmits vibrations to said vibration means.

2. The anti-nociceptive apparatus according to claim 1, wherein the vibration means comprises:
a ring portion at a proximal end, an elongated round body at a distal end, and a neck portion connecting both the ring portion and the elongated round body;
coaxial tubular portions located in the elongated round body along the longitudinal axis, provided in one or a plurality of configurations, which open to the distal end of said body and which insertably and detachably are coupled with a vibration resonance means of the handle assembly; and
a means of the ring portion to contact with the recipient's tissue and to deliver circumferential vibrations to said tissue, provided in one or a plurality of configurations, which has a range of cross-sectional thickness, a range of radius and a range of elasticity.

3. The anti-nociceptive apparatus according to claim 1, wherein the handle assembly comprises:
a tubular body, provided in one or a plurality of configurations including a compartmentalized longitudinal tubular configuration, which is connected to the vibration resonance means at a proximal end and to an electricity conduit at a distal end and which houses a vibration assembly and a control and power assembly arranged in tandem longitudinally;
the vibration assembly, provided as one or a plurality of operating devices having one or a plurality of mechanical and electronic configurations, which generates and transmits vibrations to the vibration means;
the control and power assembly, provided as one or a plurality of operating devices having one or a plurality of mechanical and electronic configurations, which provides the vibration assembly with electricity and control; and
the vibration resonance means, provided in one or a plurality of configurations, which is a proximally located device of the vibration assembly, which amplifies vibrations by resonance, which is releasably inserted in the tubular portion of the elongated round body of the vibration means and which transmits resonated vibrations to said vibration means.

4. The anti-nociceptive apparatus according to claim 3, wherein the vibration assembly comprises:
a vibrator, which is provided as one or a plurality of electromagnetic devices releasably assembled with one or a plurality of permanent magnets, which is configured to produce mechanical vibrations and which is electrically connected to the control and power assembly;
the electromagnetic device, having one or a plurality of electromagnetic configurations, which receives electric current from the control and power assembly, which is configured to generate vibrations in one or a plurality of frequencies and of one or a plurality of amplitudes and which may concurrently generate vibrations of varying frequencies; and
a means to transmit vibrations to the vibration resonance means, provided in one or a plurality of mechanical configurations, which connects one end of the vibrator to said vibration resonance means.

5. The anti-nociceptive apparatus according to claim 3, wherein the control and power assembly comprises:
a control electronics, provided in one or a plurality of electronic configurations, which comprises an electronic circuit board, an electronic switch and an instrument panel, which is electrically connected to the vibration assembly and which provides said vibration assembly with power and electronic control for frequency and amplitude of vibrations;
the electronic switch, provided as one or a plurality of operating devices having one or a plurality of mechanical and electronic configurations, which is electrically connected to the vibration assembly, the electronic circuit board and a power source, which turns on and off the vibration assembly and which directs the electronic circuit board to vary frequencies and amplitudes of vibrations by a plurality of pre-set numbers of push-to-make and push-to-break actions on said switch; and
the electronic circuit board, provided in one or a plurality of electronic configurations, which controls and modifies electric current from a power source to vary frequencies and amplitudes of vibrations of the vibrator.

6. The anti-nociceptive apparatus according to claim 2, wherein the vibration means is configured to deliver circumferential vibrations to a tissue of a recipient by contact and to surround said recipient's needle penetration site placed within the circumference.

7. The anti-nociceptive apparatus according to claim 2, wherein the vibration means is detachable from the handle assembly.

8. The anti-nociceptive apparatus according to claim 3, wherein the handle assembly is configured to shield an operator's hand from an electromagnetic field generated by the vibration assembly.

9. The anti-nociceptive apparatus according to claim 3, wherein the handle assembly is configured to reduce vibrations of the tubular body of said handle assembly.

10. The anti-nociceptive apparatus according to claim 3, wherein the vibration resonance means is configured to resonate vibrations transmitted to said vibration resonance means.

11. The anti-nociceptive apparatus according to claim 4, wherein the vibration assembly is configured to simultaneously generate vibrations of a plurality of frequencies.

12. The anti-nociceptive apparatus according to claim 4, wherein the vibration assembly is configured to be electronically controllable for frequencies and amplitudes of vibrations.

13. A method for the anti-nociceptive apparatus according to claim 1, wherein vibration encircles a needle penetration site of a recipient.

14. A method for the anti-nociceptive apparatus according to claim 3, wherein the vibration resonance means resonates vibrations transmitted from the vibration assembly and transmits said resonated vibrations to the vibration means.

15. A method for the anti-nociceptive apparatus according to claim 4, wherein the vibration assembly generates vibrations of one or a plurality of frequencies and of one or a plurality of amplitudes.
